(19) United States
(12) Patent Application Publication
(10) Pub. No.: US 2016/0058326 A1
(43) Pub. Date: Mar. 3, 2016

(54) GAIT AND MOBILITY ASSESSMENT SYSTEMS AND METHODS

(71) Applicants: Kyle N. WINFREE, Paoli, PA (US); Ingrid A. PRETZER-ABOFF, Hockessin, DE (US); Sunil K. AGRAWAL, Newark, DE (US)

(72) Inventors: Kyle N. Winfree, Flagstaff, AZ (US); Ingrid Pretzer-Aboff, Hockessin, DE (US); Sunil K. Agrawal, Newark, DE (US)

(73) Assignee: UNIVERSITY OF DELAWARE, Newark, DE (US)

(21) Appl. No.: 14/784,459

(22) PCT Filed: Apr. 14, 2014
(86) PCT No.: PCT/US14/33975

§ 371 Date: Oct. 14, 2015

Related U.S. Application Data
(60) Provisional application No. 61/811,952, filed on Apr. 15, 2013.

(51) Int. Cl. A61B 5/103; A61B 5/00

(52) U.S. Cl. CPC A61B 5/1038; A61B 5/6807; A61B 5/0004; A61B 5/7246; A61B 5/7278; A61B 2019/464

(57) ABSTRACT

Gait and mobility assessment systems and methods are disclosed. A gait and mobility assessment system comprises a shoe, a plurality of force sensors mounted on the shoe, a plurality of motion sensors mounted on the shoe, and a microprocessor affixed to the shoe. The shoe is adapted to be secured to a foot of a user such that the user may freely ambulate in an unrestricted environment. The force sensors are configured to sense forces exerted by the foot of the user. The motion sensors are configured to sense a motion of the shoe. The microprocessor is coupled to receive data from the plurality of force sensors representing the sensed forces and from the plurality of motion sensors representing the sensed motion. The microprocessor is programmed to (i) store the received data in a memory affixed to the shoe, or (ii) wirelessly transmit the received data to a remote location.

### CROSS REFERENCE TO RELATED APPLICATIONS

This application claims priority to U.S. Patent Application No. 61/811,952, entitled “GAIT TRAINING SYSTEMS AND METHODS”, filed Apr. 15, 2013, the contents of which are incorporated herein by reference in their entirety.

### FIELD OF THE INVENTION

The present invention relates generally to gait and mobility assessment systems and methods.

### BACKGROUND OF THE INVENTION

Often, patients with significant illnesses that affect the neurological system (e.g. Parkinson's disease) experience degradation in their control of motor skills. In particular, this degradation can significantly affect a patient's walking ability or gait. Patients with these degenerative neurological conditions may require specialized treatments to regain or maintain ambulatory ability. Accordingly, systems and methods are desired for monitoring and improving the gait of patients with degenerative neurological conditions.

Parkinson's disease (PD) has a profound effect on the ability of the individual to walk. The cumulative effects of decreased levels of dopamine, decreased levels of activity, and the physical environment have a potent influence on the PD person's gait. The desired outcomes of medical, surgical, and rehabilitative PD treatments are improved gait and mobility. However, current measurements of gait and mobility do not address the PD patient's daily fluctuation in gait, nor do they account for the influence of the patient's physical home and community environment.

### SUMMARY OF THE INVENTION

Aspects of the present invention are directed to gait and mobility assessment systems and methods.

In accordance with one aspect of the present invention, a gait and mobility assessment system is disclosed. The gait and mobility assessment system comprises a shoe, a plurality of force sensors mounted on the shoe, a plurality of motion sensors mounted on the shoe, and a microprocessor affixed to the shoe. The shoe is adapted to be secured to a foot of a user such that the user may freely ambulate in an unrestricted environment. The force sensors are configured to sense forces exerted by the foot of the user. The motion sensors are configured to sense a motion of the shoe. The microprocessor is coupled to receive data from the plurality of force sensors representing the sensed forces and from the plurality of motion sensors representing the sensed motion. The microprocessor is programmed to (i) store the received data in a memory affixed to the shoe, or (ii) wirelessly transmit the received data to a remote location.

In accordance with another aspect of the present invention, a method of monitoring the gait of a user is disclosed. The method includes providing a gait and mobility assessment system adapted to be secured to a foot of the user and to permit the user to freely ambulate in an unrestricted environment, receiving data from the plurality of force sensors representing the sensed forces and from the plurality of motion sensors representing the sensed motion with a microprocessor, and performing at least one of the steps of storing the received data in a memory affixed to the gait and mobility assessment system, and wirelessly transmitting the received data to a remote location.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following figures:

FIG. 1A is a diagram illustrating an exemplary gait and mobility assessment system in accordance with aspects of the present invention;

FIGS. 1B and 1C are images of the gait and mobility assessment system of FIG. 1A;

FIG. 2 is a flow chart illustrating an exemplary method for monitoring the gait of a user in accordance with aspects of the present invention;

FIG. 3 is a flow chart illustrating an exemplary method for analyzing the gait of a user in accordance with aspects of the present invention;

FIG. 4 is a graph illustrating average heel, ball, and toe forces measured during an exemplary walking movement vs. those measured during an exemplary shuffling movement; and

FIGS. 5A and 5B are graphs illustrating spectral analysis of an exemplary walking movement and an exemplary freezing-of-gait movement, respectively.

### DETAILED DESCRIPTION OF THE INVENTION

The systems and methods disclosed herein are usable to monitor a user's gait, e.g., by monitor the user's walking ability over long periods of time in unrestricted environments. The measurement of gait in a gait lab can be precise, but expensive. However, people often walk differently in the real world environment. In order to effectively assess whether a therapeutic (medical or non-medical) intervention has been effective, the disclosed systems and methods of the present invention enable one to assess the ambulation of a user in their home and community environment. Additionally, the user's mobility may be tracked. Mobility is a major goal of intervention. The disclosed systems may be used on all populations of patients, all ages of ambulatory individuals (including children).

Generally, these systems and methods involve sensing features of the movements of the user's feet, including forces, acceleration, altitude, and global positioning. It is desirable that the walking motion be performed under the user's own power, when the user is freely ambulating in an unrestricted environment. As used herein, the above phrase refers to a situation in which the user's shoe is not attached (untethered) to an external device, but is able to be worn and moved around in an unrestricted environment (e.g. the user's home, place of work, or the community). The systems and methods may be used while a user ambulates on a treadmill, or during a user's normal point-to-point ambulation over extended periods of time (e.g., the course of a day).

The systems and methods disclosed herein are particularly suitable for users with neurological disorders (e.g., Parkinson's disease, multiple sclerosis, stroke, cerebral palsy, ataxia, and other irregular and regular gait patterns). The monitoring enabled by the disclosed systems and methods may be important for measuring gait characteristics of a user, and for identifying any variability in the user's gait under a wide range of ambulating conditions, and from an analysis of this information, assisting the user in maintaining or regaining a normal, healthy gait. Alternatively, the systems and methods disclosed herein may be usable for sports science applications, for example, in order to obtain detailed information on the movement of an athlete's leg during an athletic activity.

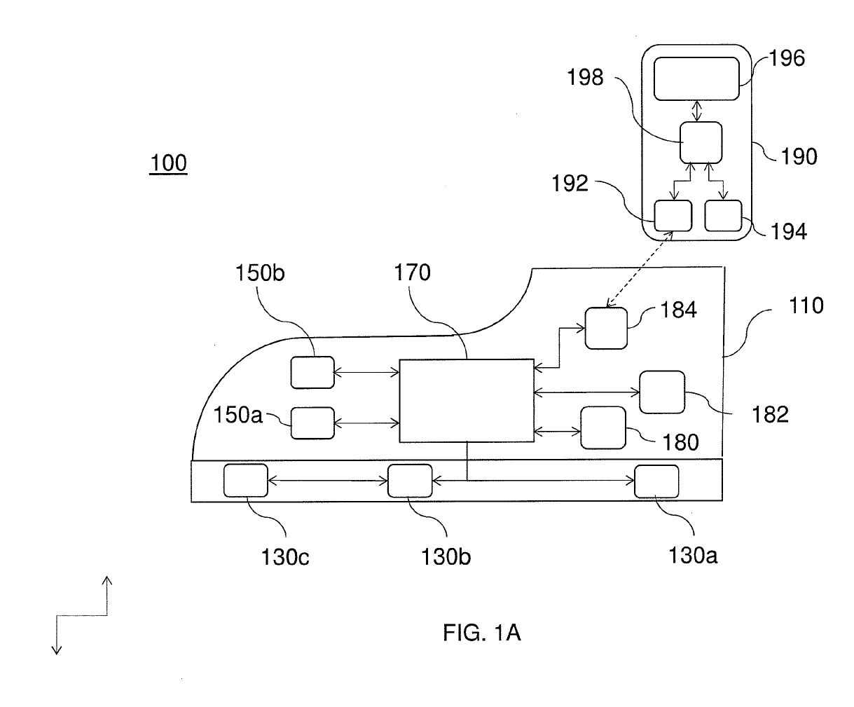

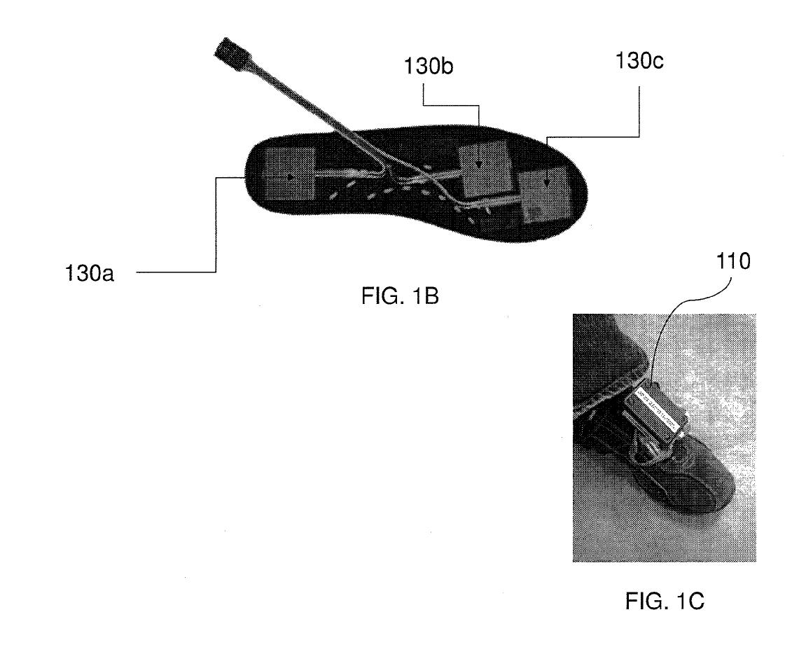

Referring now to the drawings, FIGS. 1A-1C illustrate an exemplary gait and mobility assessment system 100 in accordance with aspects of the present invention. Assessment system 100 is usable to monitor and collect data regarding the gait and mobility of a user. As a general overview, system 100 includes a shoe 110, a plurality of force sensors 130a-130c, a plurality of motion sensors 150a and 150b, and a microprocessor 170. Additional details of assessment system 100 are described herein.

Shoe 110 is adapted to be secured to the foot of the user of system 100. As used herein, the term “shoe” is not intended to refer to any particular category or style of footwear. The term “shoe” is intended to encompass any and all structures adapted to be secured to a user's foot. Shoe 110 may refer to an entire piece of footwear, or may refer to a device that forms only a portion of the user's footwear, such as an insole, for example. Shoe 110 may also refer to a device that is configured to be attached to the user's normal footwear, thereby enabling the user to ambulate in their own footwear and increasing user comfort and familiarity. Suitable structures for use as shoe 110 will be known to one of ordinary skill in the art from the description herein. An image of a shoe 110 configured for attachment to a user's footwear is shown in FIG. 1C.

System 100 desirably monitors a user's gait when the user is walking under his or her own power. Accordingly, shoe 110 is secured to the user's foot in such a way that the user can still freely ambulate in an unrestricted fashion/environment. To this end, shoe 110 is not attached to any other device or frame external to shoe 110. As long as the shoe 110 is not attached to an external device (i.e., is untethered), the user will be capable of moving around in an unrestricted environment (e.g. the user's home, place of work, or the community).

Force sensors 130a-130c are mounted on shoe 110. Force sensors 130a-130c are mounted on shoe 110 in positions to enable them to sense forces exerted by the user's foot during standing or ambulation by the user. In an exemplary embodiment, force sensors 130a-130c are mounted so as to be underneath the user's foot when shoe 110 is secured to the user's foot, e.g., in the sole of shoe 110.

Force sensors 130a-130c may be spaced from each other to enable sensing of forces exerted by different portions of the user's foot. In an exemplary embodiment, as shown in FIG. 1A, force sensors 130a-130c include a heel sensor 130a configured to sense a force exerted by the user's heel, a ball sensor 130b configured to sense a force exerted by the ball of the user's foot, and a toe sensor 130c configured to sense a force exerted by one or more of the user's toes. While three force sensors 130a-130c are illustrated in FIG. 1A, it will be understood that system 100 may include any number of force sensors. For example, the forces sensors may include a pair of ball sensors, one positioned on the medial (inner) side of the foot and one positioned on the lateral (outer) side of the foot.

Suitable sensors for use as force sensors 130a-130c include Standard 406 FSRT force sensors provided by Interlink Electronics, of Camarillo, Calif. Other suitable sensors for use as force sensors will be known to one of ordinary skill in the art from the description herein.

Motion sensors 150a and 150b are mounted on shoe 110. Motion sensors 150a and 150b are mounted on shoe 110 in positions to enable them to sense motion of shoe 110 in any direction during standing or ambulation by the user.

In an exemplary embodiment, as shown in FIG. 1A, motion sensors 150a and 150b include at least one accelerometer and at least one gyroscope. The accelerometer is preferably a three-axis accelerometer configured to sense an acceleration of shoe 110 along three distinct axes. The gyroscope is preferably a three-axis gyroscope configured to sense a rotational acceleration of shoe 110 along three distinct angular directions. While two motion sensors 150a and 150b are illustrated in FIG. 1A, it will be understood that assessment system 100 may include any number of motion sensors.

Suitable sensors for use as the accelerometer include, for example, the ADXL-335 accelerometer, provided by Analog Devices, of Norwood, Mass. Suitable sensors for use as the gyroscope include, for example, the ITG-3200 MEMS gyro provided by InvenSense Inc. of San Jose, Calif., USA. Other suitable sensors for use as motion sensors will be known to one of ordinary skill in the art from the description herein.

Microprocessor 170 controls the operation of system 100. Microprocessor 170 is affixed to shoe 110, and is electrically coupled with force sensors 130a-130c and motion sensors 150a and 150b. In particular, microprocessor 170 is coupled to receive data from force sensors 130a-130c representing the sensed forces, and to receive data from motion sensors 150a and 150b representing the sensed motion. Further, microprocessor 170 is programmed to either (i) store the received data in a memory or (ii) wirelessly transmit the received data to a remote location.

In an exemplary embodiment, microprocessor 170 is a microcontroller provided by Sparkfun, Inc. of Boulder, Colo. Other suitable data processors for use as microprocessor 170 will be known to one of ordinary skill in the art from the description herein. Additional details regarding the programming of microprocessor 170 are described herein with respect to the operation of system 100.

Gait and mobility assessment system 100 is not limited to the above components, but may include alternative or additional components, as would be understood by one of ordinary skill in the art.

Gait and mobility assessment system 100 may include a data storage component or memory (not shown) for use in storing the data generated during operation of system 100. In an exemplary embodiment, microprocessor 170 may be configured to store data received from force sensors 130a-130c and/or motion sensors 150a and 150b during ambulation by the user. Suitable data storage components include, for example, Secure Digital (SD) cards.

Gait and mobility assessment system 100 may also include an altitude sensor 180, as shown in FIG. 1A. Altitude sensor 180 is mounted on shoe 110, and is configured to sense a relative height of shoe 110. In an exemplary embodiment, microprocessor 170 is coupled to receive data from altitude sensor 180 representing the relative height, and to either store and/or transmit the received data. Suitable sensors for use as altitude sensor 180 will be known to one of ordinary skill in the art from the description herein.

Gait and mobility assessment system 100 may also include a global positioning sensor 182, as shown in FIG. 1A. Global positioning sensor 182 is mounted on shoe 110, and is configured to sense a relative position of shoe 110. In an exemplary embodiment, microprocessor 170 is coupled to receive data from global positioning sensor 182 representing the relative position, and to either store and/or transmit the received data. This information may be desirable in order to enable determination of what type environment (e.g., home, work, etc.) the user is in during a particular movement or ambulation recorded by the other sensors of system 100. Suitable sensors for use as global positioning sensor 182 will be known to one of ordinary skill in the art from the description herein.

Gait and mobility assessment system 100 may also include a first wireless communication module 184, as shown in FIG. 1A. Where wireless communication module 184 is included, microprocessor 170 is programmed to wirelessly transmit the received data from force sensors 130a-130c and motion sensors 150a and 150b to a remote location using wireless communication module 184.

Gait and mobility assessment system 100 may further comprise a wireless communication device 190 which is a separate component from shoe 110. Wireless communication device 190 comprises a second wireless communication module 192 adapted to communicate with the first wireless communication module 184 in shoe 110. In an exemplary embodiment, wireless communication device 190 comprises a cell phone or a portable digital assistant (PDA). Microprocessor 170 is configured to transmit data using first wireless communication module 184 to wireless communication device 190 via second wireless communication module 192, e.g., via a near-field or Bluetooth transmitter coupled to microprocessor 170.

Wireless communication device 190 also comprises a third wireless communication module 194 adapted to transmit data to a remote location. Wireless communication device 190 is configured to receive data from locally positioned microprocessor 170 via second wireless communication module 192, and transmits the received data to a remote location, e.g., a centralized monitoring center, via the third wireless communication module 194. Device 190 may be configured to automatically or manually transmit the data to the remote location using module 194. Such transmission may desirably allow a single centralized location to remotely monitor user data from multiple systems 100 at the same time.

The wireless communications modules 184, 192, 194 described herein may be selected based on the desired type and range of communication to be achieved by each. Where module 184 need only communicate with a locally positioned device 190, modules 184 and 192 may comprise a near-field communications transceivers. Alternatively, where module 184 must transmit the data over larger distances, it may comprise a radio-frequency of cell-phone transceiver. Likewise, where device 190 having third wireless communications module 194 is employed, module 194 may comprise a radio-frequency of cell-phone transceiver. Suitable transceivers for use as any of these communications modules will be known to one of ordinary skill in the art from the description herein. The communications modules 184, 192, 194 are preferably transceivers capable of sending and receiving signals, even if their primary function is only sending or receiving, to permit sending acknowledgements of transmissions received, and receiving acknowledgements of transmissions sent.

Wireless communication device 190 may also include a feedback component 196 and a microprocessor 198. Feedback component 196 may be configured to provide audio feedback (such as a speaker) or video feedback (such as a display screen) to a user. The feedback component 196 may provide feedback to the user based on the user's ambulation within system 100. For example, wireless communication device 190 may be configured to calculate the number of steps the user has taken based on the data from force sensors 130a-130c and motion sensors 150a and 150b, and display this number of steps to the user via the feedback component 196. Additionally, wireless communication device 190 may be configured to calculate the distance the user has walked based on data from global positioning sensor 182, and display this distance to the user via the feedback component 196. Such calculations or other analysis may be performed by microprocessor 198.

Once the data from sensors 130a-130c and 150a and 150b is received by microprocessor 170, wireless communication device 190, and/or the remote (centralized) location, it may be further processed and analyzed to identify characteristics of the user's gait. For example, the analysis described herein may be performed by microprocessor 170 or by microprocessor 198. Examples of the types of analysis that may be performed will be discussed in greater detail herein.

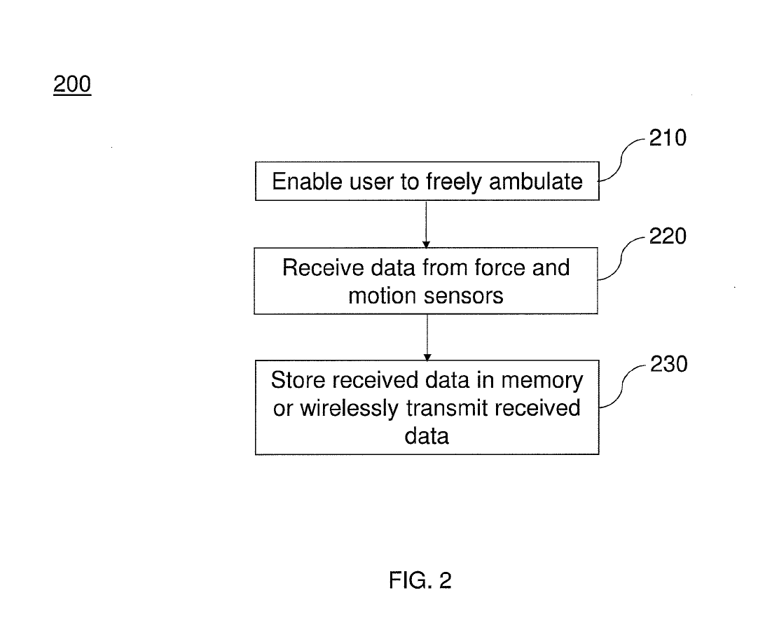

FIG. 2 shows an exemplary method for monitoring the gait of a user 200 in accordance with aspects of the present invention. As a general overview, method 200 includes enabling a user to ambulate and receiving data from sensors regarding the user's ambulation. Additional details of method 200 are described herein with respect to the components of gait and mobility assessment system 100.

In step 210, a user is enabled to freely ambulate. In an exemplary embodiment, a user is enabled to ambulate with gait and mobility assessment system 100 secured to the user's foot. The user is enabled to freely ambulate in an unrestricted environment. As set forth above, this means that shoe 110 is not attached to any other device or frame external to shoe 110. Gait and mobility assessment system 100 includes a plurality of force sensors 130a-130c configured to sense forces exerted by the user's foot during step 210 and a plurality of motion sensors 150a and 150b configured to sense a motion of shoe 110 during step 210.

Because shoe 110 may lack any active (e.g. feedback components), step 210 may be performed over extended lengths of time without concerns relating to power consumption. For example, step 210 may extend for hours or even days, while data is continuously collected by force sensors 130a-130c and motion sensors 150a and 150b.

In step 220, data is received from sensors 130a-130c and 150a and 150b. In an exemplary embodiment, the data collected by force sensors 130a-130c and motion sensors 150a and 150b during step 210 is received by microprocessor 170. This data represents the forces and motions experienced by shoe 110 during ambulation.

In step 230, microprocessor 170 performs at least one additional step with the received data. In particular, microprocessor 170 either stores the data in a memory affixed to system 100, or wirelessly transmits the received data to a remote location. In an exemplary embodiment, system 100 includes a data storage component, and microprocessor 170 is programmed to continuously store the data in the data storage component over the duration of the monitoring period (e.g., 1-30 days). In another exemplary embodiment, system 100 includes a wireless communication device, and microprocessor 170 is programmed to periodically wirelessly transmit the received data to the remote location via the wireless communication device 190. Such periodic wireless transmission may occur, for example, hourly or daily.

Method 200 is not limited to the above steps, but may include alternative steps and additional steps, as would be understood by one of ordinary skill in the art from the description herein.

For one example, the data stored or transmitted by microprocessor 170 may be analyzed to identify characteristics (such as variability) of the user's gait. Such analysis may be conducted at the remote (or centralized) location using the data received by microprocessor 170, which may either be obtained directly from the data storage component in system 100 or wirelessly via wireless communication device 190. The form of the analysis may be selected based on the types of characteristics to be identified for the user's gait.

In one embodiment, the data received from the plurality of force and motion sensors may be analyzed to determine a variability in the shape or rate of the user's gait. Such variability may take the form, for example, of changes in step length, step duration or frequency, duration of different step phases, or any combination thereof.

In another embodiment, the data received from the plurality of force and motion sensors may be analyzed to determine a variability in the user's gait dependent on when the user is ambulating. For example, the data may be analyzed to determine a variability between whether the user is walking or whether the user is performing some other motion (e.g., standing still or sitting).

For an embodiment that includes an altitude sensor as discussed above, the data received from the altitude sensor may be analyzed to determine whether the user is ambulating upwards or downwards. This information may be desirable for determining the effect of ambulating upwards or downwards on the user's gait.

For an embodiment that includes a global positioning sensor as discussed above, the data received from the global positioning sensor may be analyzed to identify variability in the user's gait that is dependent on the environment in which the user is ambulating. This information may be desirable for determining changes in the user's gait based on whether the user is ambulating in home or in public, for example.

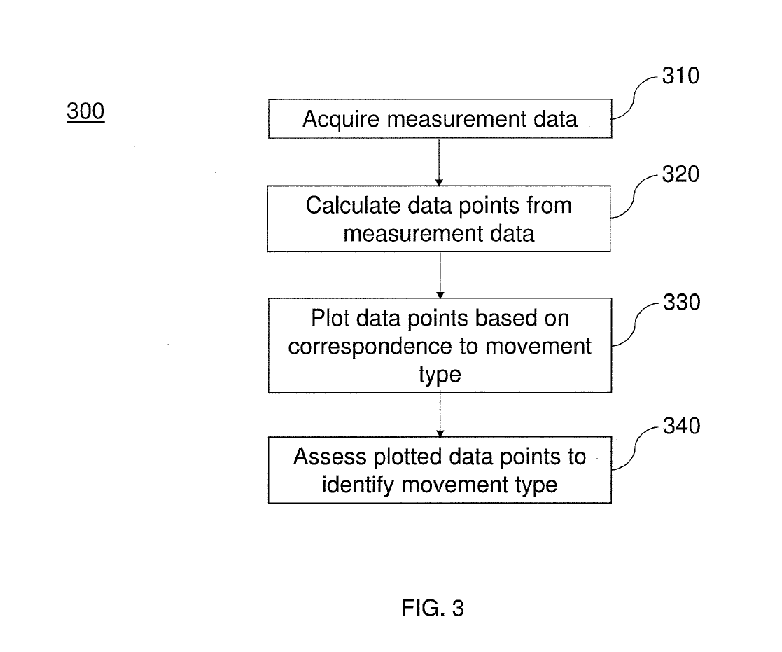

FIG. 3 shows an exemplary method 300 for analyzing the data obtained during method 200. Method 300 may be usable to determine what type of movement is being performed by the user of system 100. As a general overview, method 300 includes acquiring measurement data, calculating data points, plotting the data, and assessing movement type. Additional details of method 200 are described herein with respect to the components of gait and mobility assessment system 100.

Prior to the enumerated steps, data points for each user are precalculated for various movement types of interest. Movement types of interest may include, for example, walking, standing, sitting, shuffling, freezing-of-gait, jogging, running, jumping, climbing stairs, or transitions between any of these movement types (particularly sitting-to-standing transitions and vice versa). These data points may be acquired while the user is moving in a laboratory environment, so that the type of movement can be visually identified and associated with the data points calculated during each predetermined movement type. However, when the user is freely ambulating in an unrestricted (and uncontrolled) environment (as during method 200), it cannot be predetermined what type of movement is being performed. Thus, the movement type must be determined from the measurement data acquired from force sensors 130a-130c and motion sensors 150a and 150b.

A discriminatory function can be used to identify gait. As one example, this can be done with logistic regression. However, other related methods may be used that would be known to one of ordinary skill in the art from the description herein.

In step 310, measurement data is acquired. In an exemplary, force sensors 130a-130c and motion sensors 150a and 150b acquire measurement data during ambulation of the user, as described above with respect to method 200. The measurement data is then input to either microprocessor 170, wireless communication device 190, and/or a remote centralized monitoring center, depending on where the analysis is being conducted. The analysis described with respect to method 300 may be performed in any of these locations.

In an exemplary embodiment, the measurement data includes 10 distinct measurements; four force measurements and six acceleration measurements. The four force measurements are from heel sensor 130a, lateral and medial ball sensors 130b, and toe sensor 130c. The six acceleration measurements represent the six axes (three linear and three angular) from motion sensors 150a and 150b. New values of each of these measurements may be acquired based on the measurement or sampling rates of the respective sensors. For example, measurements may be acquired at approximately 60-70 Hz, based on the sensors used.

In step 320, data points are calculated. The data points may be calculated to represent the variance in the measurement data acquired from the sensors. In an exemplary embodiment, 14 data points are calculated for each category of measurement data (resulting in approximately 140 data points). The 14 data points may comprise the mean, standard deviation, root mean square (RMS), and eleven fast Fourier transforms (FFTs) for each set of measurements. The eleven FFTs are broken up by frequency in 1 Hz increments (from 0.5 to 11.5 Hz). These data points desirably represent the variance in each measurement category over the time during which the user is ambulating.

In step 330, the data points are plotted. The data points calculated in step 320 are plotted based on how they correspond to various types of movement for which the predetermined data was acquired. In an exemplary embodiment, a best fit line is predetermined for the previously acquired data points for each movement type. The calculated data points are then plotted in two bands based on whether or not they correspond to the best fit line for a particular movement type. If the calculated data points are sufficiently close to the best fit line, they are plotted in one band representing correspondence to the movement type; if the calculated data points are not sufficiently close to the best fit line, they are plotted in another band representing a lack of correspondence. The data points may be plotted against the best fit line for each movement type of interest.

In step 340, the plotted data points are assessed to determine the type of movement being performed by the user. In an exemplary embodiment, after all data points are plotted, an assessment is made as to how many of the 140 data points fall within the correspondence band (as opposed to the non-correspondence band). If the number of data points in the correspondence band is above a predetermined threshold, then it is assessed that the user was performing that type of movement. For example, if at least 75% of the data points correspond to the predetermined best fit line for “walking” data points for the user, it can be assessed that the user was walking during this time period. The predetermined threshold (e.g., 75%) may be selected or adjusted based on the desired specificity or sensitivity of the analysis process.

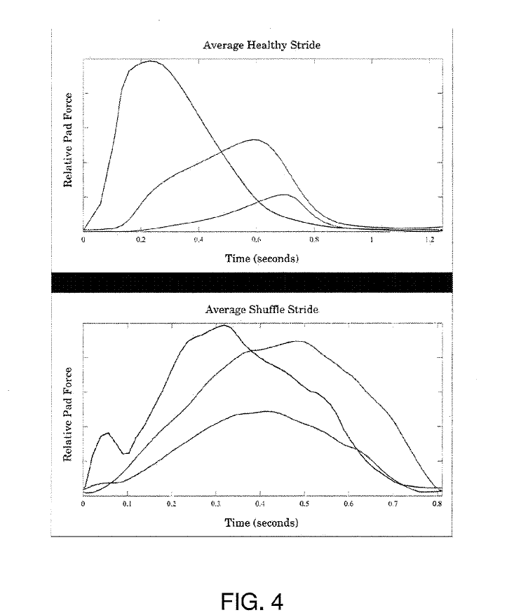

After the above analysis is completed and the type of movement (e.g., walking) is identified, the measurements can be analyzed to identifying characteristics about the user's walking gait. Such characteristics can include, for example, the forces experienced during heel strikes, ball strikes, and toe strikes during the user's walk. Averages and standard deviations of these forces can be taken to approximately the normal forces experienced during the user's walk. Such information may be useful to identify improvements or degradations in the user's gait. FIG. 4 illustrates an exemplary graph of average heel, ball, and toe forces measured during a walking movement vs. those measured during a shuffling movement of an exemplary user.

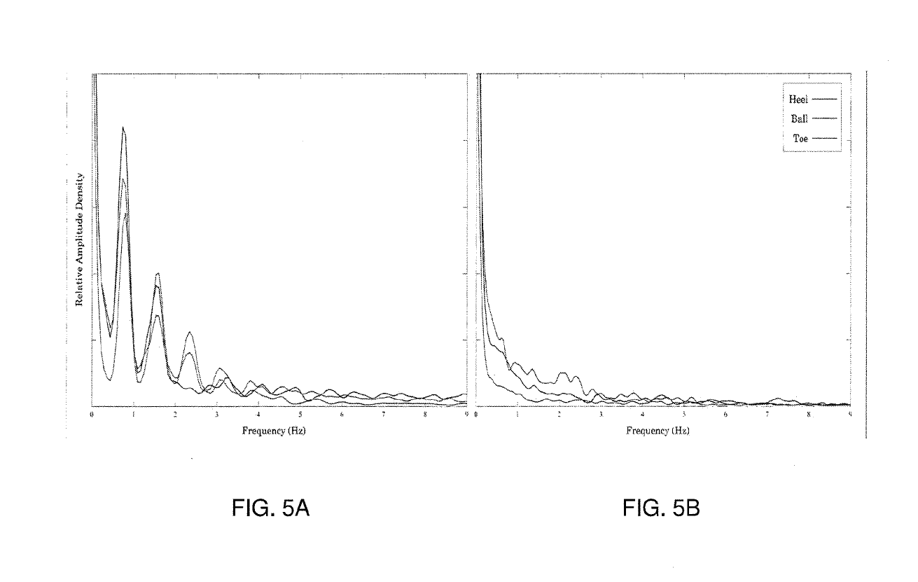

Additionally, spectral analysis of the data may be performed to identify the common frequencies experience by the user during a walking movement. FIGS. 5A and 5B illustrate graphs of spectral analysis of an exemplary walking movement and an exemplary freezing-of-gait movement, respectively, for an exemplary user. As shown in FIG. 5A, a normal walking movement shows a high spectral density at both 0.77 Hz and 1.59 Hz on the heel, ball, and toe sensors. An additional mode is visible on the ball and toe sensors at 2.32 Hz. These spikes in frequency composition are not present in the FOG sample, shown in FIG. 5B. Spectral response is limited to less than 2.5 Hz, and is nearly absent at higher frequencies.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A gait and mobility assessment system comprising:
a shoe adapted to be secured to a foot of a user such that the user may freely ambulate in an unrestricted environment;
a plurality of force sensors mounted on the shoe, the force sensors configured to sense forces exerted by the foot of the user;
a plurality of motion sensors mounted on the shoe, the motion sensors configured to sense a motion of the shoe; and
a microprocessor affixed to the shoe, the microprocessor coupled to receive data from the plurality of force sensors representing the sensed forces and from the plurality of motion sensors representing the sensed motion, and the microprocessor programmed to store the received data in a memory affixed to the shoe or wirelessly transmit the received data to a remote location.

2. The gait and mobility assessment system of claim 1, wherein the shoe is not attached to any other device external to the shoe.

3. The gait and mobility assessment system of claim 1, wherein the plurality of force sensors comprises at least a heel sensor configured to sense a force exerted by a heel of the foot of the user, a ball sensor configured to sense a force exerted by a ball of the foot of the user, and a toe sensor configured to sense a force exerted by one or more toes of the foot of the user.

4. The gait and mobility assessment system of claim 1, wherein the plurality of motion sensors comprises at least one accelerometer configured to sense an acceleration of the shoe along three different axes.

5. The gait and mobility assessment system of claim 4, wherein the plurality of motion sensors further comprises at least one gyroscope configured to sense an acceleration of the shoe along three different angular directions.

6. The gait and mobility assessment system of claim 1, further comprising an altitude sensor mounted on the shoe, the altitude sensor configured to sense a relative height of the shoe.

7. The gait and mobility assessment system of claim 1, further comprising a global positioning sensor mounted on the shoe, the global positioning sensor configured to sense a relative position of the shoe.

8. The gait and mobility assessment system of claim 1, further comprising a wireless communication device, wherein the microprocessor is programmed to wirelessly transmit the received data to the remote location via the wireless communication device.

9. A method of monitoring a gait of a user, the method comprising the steps of:
providing a gait and mobility assessment system adapted to be secured to a foot of the user and to permit the user to freely ambulate in an unrestricted environment, the gait and mobility assessment system comprising:
a plurality of force sensors configured to sense forces exerted by the foot of the user, and
a plurality of motion sensors configured to sense a motion of the shoe;
receiving data from the plurality of force sensors representing the sensed forces and from the plurality of motion sensors representing the sensed motion with a microprocessor; and
performing at least one of the steps of:
storing the received data in a memory affixed to the gait and mobility assessment system, and
wirelessly transmitting the received data to a remote location.

10. The method of claim 9, wherein the performing step comprises wirelessly transmitting the received data to the remote location via a wireless communication device

11. The method of claim 9, further comprising the step of analyzing the received data to identify a variability in the gait of the user.

12. The method of claim 11, wherein the analyzing step comprises analyzing the received data to identify a variability in a shape or rate of the gait of the user.

13. The method of claim 12, wherein the analyzing step comprises analyzing the received data to identify a variability in the gait of the user dependent on when the user is ambulating.

14. The method of claim 11, wherein the analyzing step comprises analyzing data from an altitude sensor of the gait and mobility assessment system to identify a variability in the gait of the user dependent on whether the user is ambulating upwards or downwards.

15. The method of claim 11, wherein the analyzing step comprises analyzing data from a global positioning sensor of the gait and mobility assessment system to identify a variability in the gait of the user dependent on an environment in which the user is ambulating.

16. The method of claim 11, wherein the analyzing step comprises:
calculating data points from the received data from the plurality of force sensors and the plurality of motion sensors;
plotting the data points based on correspondence to one or more of a plurality of types of movement; and
assessing the plotted data points to identify a type of movement.

17. The method of claim 16, wherein the calculating step comprises:
calculating one or more of the mean, standard deviation, root mean square, and spectral variance of the received data from the plurality of force sensors and the plurality of motion sensors.

18. The method of claim 16, wherein the plotting step comprises:
predetermining a best fit line for each of the plurality of types of movement; and
plotting the data points into a plurality of bands based on correspondence to the best fit line.

19. The method of claim 18, wherein the assessing step comprises:
identifying a type of movement by assessing whether a number of data points in a first of the plurality of bands exceeds a predetermined threshold.
